(19) United States
(12) Patent Application Publication
(10) Pub. No.: US 2012/0165589 A1
(43) Pub. Date: Jun. 28, 2012

(54) A PROCESS FOR THE DEHYDRATION OF ETHANOL TO PRODUCE ETHENE

(75) Inventor: Stephen Roy Partington, East Yorkshire (GB)

(73) Assignee: BP p.l.c., London (GB)

(21) Appl. No.: 13/379,268

(22) PCT Filed: Jun. 10, 2010
(86) PCT No.: PCT/GB2010/001142

§ 371 Date: Mar. 13, 2012

(30) Foreign Application Priority Data
Jun. 19, 2009 (EP) 09251608.7

(51) Int. Cl. C07C 1/20

(52) U.S. Cl. 585/639

(57) ABSTRACT

The present invention relates to a process for the production of ethylene, from a feedstock comprising ethanol, in the presence of a phosphotungstic acid catalyst.

The present invention relates to a process for the production of ethylene from a feedstock comprising ethanol in the presence of a phosphotungstic acid catalyst.

Ethylene is an important commodity chemical and monomer which has traditionally been produced by steam or catalytic cracking of hydrocarbons derived from crude oil. However, as crude oil is a finite resource, there is interest in finding alternative, economically viable, methods for producing ethylene which can use feedstocks not derived from crude oil.

In recent years the search for alternative feedstock materials for the production of ethylene has led to the production of ethylene from alcohols, such as methanol and ethanol, which can be produced by the fermentation of, for example, sugars, starches and/or cellulosic materials, or alternatively may be produced from synthesis gas.

U.S. Pat. No. 5,177,114 discloses a process for the conversion of natural gas to gasoline grade liquid hydrocarbons and/or olefin(s) by converting the natural gas to a synthesis gas, and converting the synthesis gas to crude methanol and/or dimethyl ether and further converting the crude methanol/dimethyl ether to gasoline and olefin(s).

U.S. Pat. No. 5,817,906 describes a process for producing light olefin(s) from a crude oxygenate feedstock comprising alcohol and water. The process employs two reaction stages. Firstly, the alcohol is converted, using reaction with distillation, to an ether. The ether is then subsequently passed to an oxygenate conversion zone containing a metalaluminosilicate catalyst to produce a light olefin stream.

U.S. Pat. No. 4,398,050 describes the synthesis of a mixed alcohol stream and purification to give a mixture of ethanol and propanol which is subsequently dehydrated at 0.05-0.1 MPa, 350-500° C. (example 1). U.S. Pat. No. 4,398,050 specifically discloses Al₂O₃, SiO₂, TiO₂, AlPO₄ and Ca₃(PO₄)₂ as examples of suitable dehydration catalysts, with alkalized aluminium oxide or calcium phosphate being disclosed as preferred catalysts.

EP 1792885 discloses a process for the production of ethylene from a feedstock comprising ethanol. Catalysts based on heteropolyacids are disclosed as being suitable for the dehydration of the ethanol feedstock.

WO 2008/138775 A1 discloses a process for the dehydration of one or more alcohols, which process comprises contacting one or more alcohols in the presence of one or more ethers with a supported heteropolyacid catalyst.

WO 2008/062157 A1 discloses a supported heteropolyacid catalyst; a process for producing alkenes from oxygenates in the presence of said catalyst; and, the use of said catalyst in a process for producing alkenes from oxygenates at a higher productivity whilst reducing the formation of alkanes.

One of the main disadvantages of dehydrating a feedstock comprising ethanol to produce ethylene is the co-formation of C₄ compounds (e.g. butene and butane); this is because C₄ compounds are known to add significantly to the complexity and cost of producing a purified ethylene product (i.e. one that is suitable for polymer manufacture). For example, the industrially practiced catalytic cracking of hydrocarbon feedstocks to produce olefins for polymer manufacture is a capital intensive process with a significant proportion of the cost involved in removing C₄ compounds from the olefin product. Dehydration of ethanol to ethylene has been commercially practiced, in countries such as Brazil and India, on a small scale at high conversion per pass; it is shown to be a selective process but still produces unacceptable levels of C₄ compounds in order for the ethylene to be used to produce polyethylene directly.

It is an object of the present invention to provide an improved process, in terms of C₄ selectivity, for the production of ethylene from a feedstock comprising ethanol in the presence of a heteropolyacid catalyst.

The present invention thus provides a process for the preparation of ethylene comprising contacting a feedstock comprising ethanol with a supported phosphotungstic acid catalyst at a temperature in the range of from 210° C. to 270° C. and a pressure in the range of from 1.5 MPa to 2.5 MPa.

Preferably, the supported phosphotungstic catalyst used in the process of the present invention is a supported phosphotungstic acid catalyst, wherein the performance of the phosphotungstic acid catalyst under test Conditions A satisfies the following inequality:

$$\frac{\begin{matrix}Selectivity towards C 4 \\ compounds in ethylene ( ppmw )\end{matrix}}{ppmw} \leq \frac{0.6257 \times Ethylene productivity ( g / l / hr )}{g / l / hr} \qquad$$

where test Conditions A are:
a tubular plug flow reactor having an internal reactor diameter of 4.2 mm containing a catalyst volume of about 1 cm³;
a catalyst particle size of from 125 to 180 μm;
a phosphotungstic acid loading in the range of from 270 to 295 g of phosphotungstic acid per kg catalyst;
a temperature of 240° C. and a pressure of 2 MPa; and
feed flow rates as follows: ethanol (1.724 g/hr), diethylether (3.417 g/hr), water (0.080 g/hr), nitrogen (1.001 g/hr) and methane (0.032 g/hr).

The present invention further provides the use of a supported phosphotungstic acid in a process for the preparation of ethylene from a feedstock comprising ethanol, for providing a reduced selectivity toward C4 hydrocarbon compounds compared to when silicotungstic acid based catalysts are used under the same process conditions.

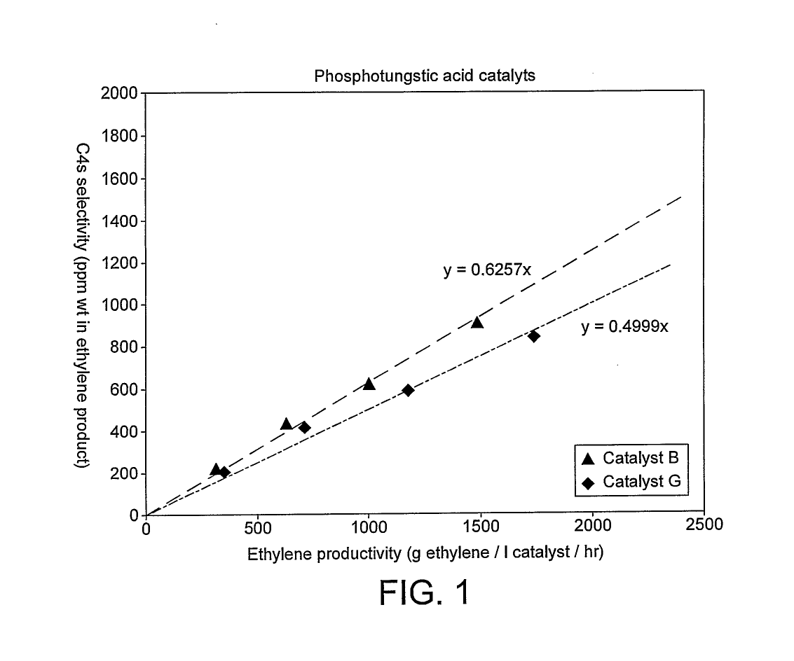

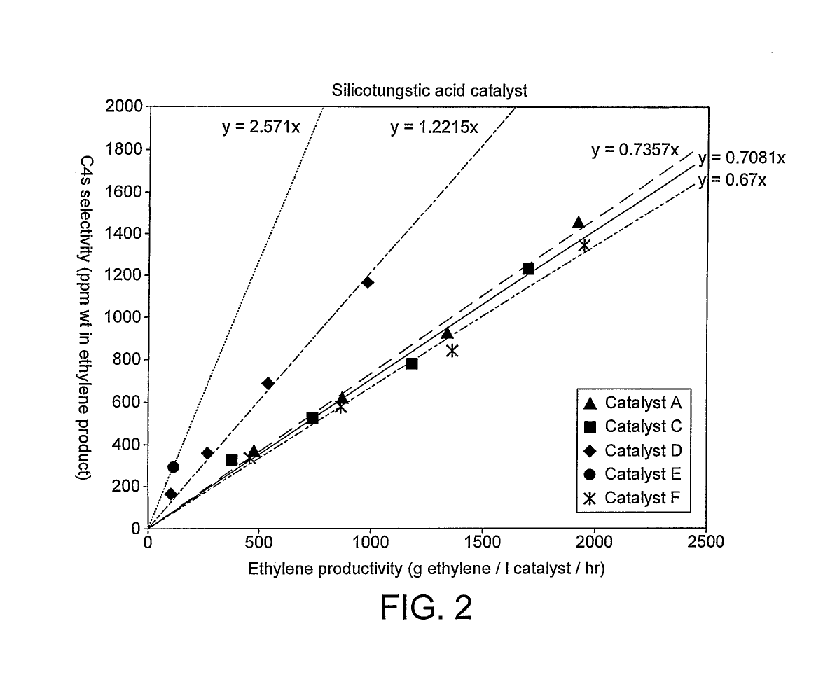

FIGS. 1 and 2 are graphical representations of C4 selectivity (ppmw in ethylene product) versus ethylene productivity (ethylene (g)/catalyst(1)/hr) for the supported phosphotungstic acid catalysts and supported silicotungstic acid catalysts of the examples.

The process of the present invention provides a method for the production of ethylene from a feedstock comprising ethanol, and proceeds via the dehydrogenation of alcohols, e.g. ethanol, and optionally ethers, e.g. diethyl ether, present in said feedstock.

The dehydration of the feedstock according to the present invention is believed (Chem. Eng Comm. 1990 vol 95 pp 27-39 C. L. Chang, A. L. DeVera and D. J. Miller) to proceed by either the direct dehydration to olefin(s) and water;

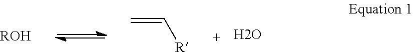

or via an ether intermediate;

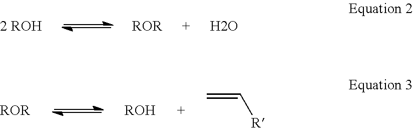

where R is an ethyl group. R′ is hydrogen.

The direct conversion of the ether to two moles of olefin and water has also been reported (Chem. Eng. Res and Design 1984 Vol 62 pp 81-91).

All of the reactions shown above are typically catalysed by Lewis and/or Bronsted acids. Equation 1 shows the endothermic direct elimination of alcohol to olefin(s) and water; competing with Equation 1 are Equations 2 and 3, i.e. the exothermic etherification reaction (Equation 2), and the endothermic elimination of ether(s) to produce olefin(s) and alcohol (Equation 3). However the dehydration reaction of alcohols to olefin(s) is overall said to be endothermic.

The supported phosphotungstic catalyst used in the process of the present invention is typically a supported phosphotungstic acid catalyst, wherein the performance of the phosphotungstic acid catalyst under test Conditions A satisfies the following inequality:

$$\frac{\begin{matrix}Selectivity towards C 4 \\ compounds in ethylene ( ppmw )\end{matrix}}{ppmw} \leq \frac{0.6257 \times Ethylene productivity ( g / l / hr )}{g / l / hr}$$

where test Conditions A are:
a tubular plug flow reactor having an internal reactor diameter of 4.2 mm containing a catalyst volume of about 1 cm³;
a catalyst particle size of from 125 to 180 μm;
a phosphotungstic acid loading in the range of from 270 to 295 g of phosphotungstic acid per kg catalyst;
a temperature of 240° C. and a pressure of 2 MPa; and
feed flow rates as follows: ethanol (1.724 g/hr), diethylether (3.417 g/hr), water (0.080 g/hr), nitrogen (1.001 g/hr) and methane (0.032 g/hr).

By the term “phosphotungstic acid” used herein, it is meant a heteropolyacid containing phosphorus and tungsten atoms. Included within the term “phosphotungstic acid” used herein, are the free acids, and the alkali, alkali earth, ammonium, bulky cation partial salts, and/or metal partial salts of the phosphotungstic acids.

Typically, each anion of the phosphotungstic acid comprises 12-18 oxygen-linked tungsten atoms, known as the peripheral atoms, surrounding one or more central phosphorus atom(s) in a symmetrical manner.

Preferably, the supported phosphotungstic acid catalysts used in the process of the present invention contains one or more phosphotungstic acid(s) having a molecular weight in the range of from 700 to 8500, more preferably in the range of from 2800 to 6000. Such supported phosphotungstic acid catalysts may also contain dimeric complexes of phosphotungstic acids.

Suitable phosphotungstic acids include Keggin, Wells-Dawson and Anderson-Evans-Perloff phosphotungstic acids. Specific examples of suitable phosphotungstic acids include:
18-tungstophosphoric acid—H₆[P₂W₁₈O₆₂].xH₂O
12-tungstophosphoric acid—H₃[PW₁₂O₄₀].xH₂0
   and partial salts, or mixtures thereof.

Examples of partial salts of phosphotungstic acids include:
Monopotassium tungstophosphate—KH₂[PW₁₂O₄₀].xH₂O
Monoammonium tungstophosphate—[NH₄]H₂[PW₁₂O₄₀].xH₂O
Monosodium tungstophosphate—NaH₂[PW₁₂O₄₀].xH₂O
Monocesium tungstophosphate—CsH₂[PW₁₂O₄₀].xH₂O
Monopotassium salt of 18-tungstophosphoric acid—KH₅[P₂W₁₈O₆₂].xH₂O
Monoammonium salt of 18-tungstophosphoric acid—[NH₄]H₅[P₂W₁₈O₆₂].xH₂O
Monosodium salt of 18-tungstophosphoric acid—NaH₅[P₂W₁₈O₆₂].xH₂O
Monocesium salt of 18-tungstophosphoric acid—CsH₅[P₂W₁₈O₆₂].xH₂O

In addition to the use of single phosphotungstic acids and partial salts thereof in the supported phosphotungstic acid catalysts used in the process of the present invention, mixtures of two or more different phosphotungstic acids and/or partial salts thereof may also be used.

The preferred phosphotungstic acid for use in the supported phosphotungstic acid catalysts used in the process of the present invention is:
12-tungstophosphoric acid—H₃[PW₁₂O₄₀].xH₂O

The supported phosphotungstic acid catalyst(s) may be conveniently prepared by dissolving the chosen phosphotungstic acid in a suitable solvent and impregnating a suitable support material with the phosphotungstic acid solution. Suitable solvents for dissolving the phosphotungstic acid include polar solvents such as water, ethers, alcohols, carboxylic acids, ketones and aldehydes and mixtures thereof; water, ethanol and mixtures thereof are the most preferred solvents; conveniently, the solvent used is water. The resulting phosphotungstic acid solution preferably has a phosphotungstic acid concentration that is in the range of from 10 to 80 wt %, more preferably in the range of from 20 to 70 wt % and most preferably in the range of from 30 to 60 wt %. The method of impregnation used to prepare the supported phosphotungstic acid catalyst is not limited, however, wet impregnation (i.e. preparation using an excess volume of phosphotungstic acid solution relative to pore volume of support) is the preferred method.

The supported phosphotungstic acid catalyst may be modified by: forming partial salts of phosphotungstic acid in the, typically aqueous, impregnation solution either prior to, or during, the impregnation; by subjecting the support or the supported phosphotungstic acid to prolonged contact with a solution of a suitable metallic salts; or, by addition of phosphoric acid and/or other mineral acids to the impregnation solution.

When the partial salt of the phosphotungstic acid is insoluble, it is preferred to impregnate the catalyst with the phosphotungstic acid and then to titrate with the salt precursor. Other techniques such as vacuum impregnation may also be employed.

The impregnated support may then optionally be washed and dried prior to use. The washing and drying of the impregnated support may be achieved using any method known in the art. For example, the impregnated support may conveniently be dried in an oven at elevated temperature; for example this may typically be conducted at 130° C. with a nitrogen flow for 16 hours and then cooled down to room temperature.

The amount of phosphotungstic acid in the supported phosphotungstic catalyst is preferably at least 10 wt %, more preferably at least 15 wt %, even more preferably at least 20 wt %, and most preferably at least 25 wt %; and preferably at most 80 wt %, more preferably at most 70 wt %, even more preferably at most 60 wt %, and most preferably at most 50 wt %, based on the total weight of the supported phosphotungstic acid catalyst.

The weight of the catalyst on drying and the weight of the support used, may be used to obtain the weight of the acid on the support, by subtracting the latter from the former, giving the catalyst loading as a ‘g phosphotungstic acid/kg catalyst’ term. The catalyst loading in ‘g phosphotungstic acid/litre support’ can also be calculated by using the known, or measured, bulk density of the support. Thus, the preferred catalytic loading of phosphotungstic acid is in the range of from 100 to 800 g of phosphotungstic acid/kg catalyst, more preferably in the range of from 150 to 700 g of phosphotungstic acid/kg catalyst, even more preferably in the range of from 200 to 600 g of phosphotungstic acid/kg catalyst, and most preferably in the range of from 250 to 500 g of phosphotungstic acid/kg catalyst.

According to a preferred embodiment of the present invention, the average phosphotungstic acid loading per surface area of the supported phosphotungstic acid catalyst is more than 0.1 micro moles/m².

It should be noted that the oxidation and hydration states of the phosphotungstic acids stated herein, only apply to the phosphotungstic acid before it is impregnated onto the support.

According to a preferred embodiment of the present invention, the amount of chloride present in, or on, the supported phosphotungstic acid catalyst is less than 40 ppmw, more preferably less than 25 ppmw, and most preferably less than 20 ppmw.

The support material used in the supported phosphotungstic acid catalyst can be any suitable support material known in the art. Suitable support materials for the supported phosphotungstic acid catalyst include, but are not limited to, mordenites (e.g. montmorillonite), clays, bentonite, diatomous earth, titania, activated carbon, alumina, silica-alumina, silica-titania cogels, silica-zirconia cogels, carbon coated alumina, zeolites, zinc oxide, flame pyrolysed oxides. Supports can be mixed, neutral or weakly basic oxides. Silica supports are preferred, such as silica gel supports and supports produced by the flame hydrolysis of SiCl₄.

Preferably, the supports used to prepare the supported phosphotungstic acid catalyst are substantially free of extraneous metals, or elements, which could adversely affect the catalytic activity of the supported phosphotungstic catalyst. Thus, any impurities that may be present in the support material preferably amounts to less than 1% w/w, more preferably less than 0.60% w/w, and most preferably less than 0.30% w/w. Thus, in a preferred embodiment, the support material used is silica having a purity of at least 99% w/w.

The pore volume of the support is preferably more than 0.50 ml/g and is more preferably more than 0.8 ml/g.

Examples of silica supports suitable for use in preparing a supported phosphotungstic catalyst include, but are not limited to: Grace Davison Davicat® Grade 57, Grace Davison Davicat® 1252, Grace Davison Davicat® SI 1254, Fuji Silysia CariAct® Q15, Fuji Silysia CariAct® Q10, Degussa Aerolyst® 3045 and Degussa Aerolyst® 3043.

The form of the catalyst support is not critical to the process of the present invention. Suitable catalyst supports may be in a powder form or a particulate form (for example: a granular form; a pelletised form; a spherical form; or in the form of an extrudate or shaped particles).

If the catalyst support is in a particulate form, the average diameter of the support particles is typically in the range of from 2 to 10 mm, preferably 3 to 6 mm. However, these particles may be crushed and sieved to smaller sizes of, for example, 0.5-2 mm, if desired.

The average pore radius (prior to impregnation with phosphotungstic acid) of the support is preferably in the range of from 10 to 500 Å, more preferably in the range of from 30 to 175 Å, even more preferably in the range of from 50 to 150 Å, and most preferably in the range of from 60 to 120 Å.

The BET surface area of the support prior to impregnation is preferably in the range of from 50 to 600 m²/g, more preferably in the range of from 150 to 400 m²/g.

Prior to impregnation, the support preferably has an average single particle crush strength of at least 1 kg force, more preferably at least 2 kg force, even more preferably at least 6 kg force, and most preferably at least 7 kg force.

Prior to impregnation, the support preferably has a bulk density of at least 380 g/l, more preferably at least 395 g/l.

The single particle crush strength referred to herein is the crush strength determined by using a Mecmesin force gauge, which measures the minimum force necessary to crush a particle between parallel plates. The crush strength is based on the average of that determined for a set of at least 25 catalyst particles.

The BET surface area, pore volume, pore size distribution and average pore radius referred to herein are calculated from the nitrogen adsorption isotherm determined at 77 K using a Micromeritics TRISTAR 3000 static volumetric adsorption analyser. The procedure used is an application of British Standard methods BS4359: Part 1:1984 ‘Recommendations for gas adsorption (BET) methods’ and BS7591:Part 2:1992, ‘Porosity and pore size distribution of materials’—Method of evaluation by gas adsorption. The resulting data is reduced using the BET method (over the pressure range 0.05-0.20 P/Po) and the Barrett, Joyner & Halenda (BJH) method (for pore diameters of 20-1000 Å) to yield the surface area and pore size distribution respectively.

Suitable references for the above data reduction methods are Brunauer, S, Emmett, P H, & Teller, E, J. Amer. Chem. Soc. 60, 309, (1938) and Barrett, E P, Joyner, L G & Halenda P P, J. Am Chem. Soc., 1951 73 373-380.

For the purpose of the above analytical measurements, samples of the supports and supported phosphotungstic acid catalysts are out gassed for 16 hours at 120° C. under a vacuum of 5×10⁻³ Torr.

In one embodiment of the present invention, the catalyst support may first be treated with a fluorinating agent; it is believed that treating the support with a fluorinating agent may make the support more inert and/or acidic and thus may lead to improved selectivity and/or effectiveness of the supported catalyst during the process of the present invention.

It has unexpectedly been observed that the selectivity toward C₄ compounds in the dehydration of a feedstock comprising ethanol can be reduced by using a supported phosphotungstic acid catalyst in the process compared to when a supported silicotungstic acid catalysts is used in the same process. The applicants have also unexpectedly found that by using a supported phosphotungstic acid catalyst, as described hereinabove, for the dehydration of feedstock comprising ethanol, it is also possible to achieve a higher selectivity towards ethylene compared to silicotungstic acid catalysts.

The preferred ethylene productivity for the process of the present invention is more than 250 (g/l/hr), preferably more than 500 (g/l/hr) and most preferably is more than 750 (g/l/hr), where ethylene productivity is defined as being: weight of ethylene (in grams)/volume of catalyst (in litres)/hour.

The process according to the present invention may be carried out in any vessel or reactor that is suitable for performing an alcohol dehydration reaction. Suitable reactor designs include those capable of handling heat fluxes such as fixed bed, fluidised bed, multi-tubular and multiple fixed bed reactors with inter-stage heaters.

Since the dehydration of alcohol is an endothermic reaction, the feedstock entering the reactor may also be heated to a temperature which is above the reaction temperature in order to provide an additional source of heat. Optionally, in order to improve heat management in some of the above-mentioned reactor designs, additional preheated feedstock may be injected at multiple points in the reactor bed.

Typically, the operating conditions under which the process of the present invention is operated are such that the dehydration process is always operated in a vapour phase state. Preferably, the operating pressure of the process of the present invention is at least 0.1 MPa, more preferably at least 0.2 MPa, below the dew point pressure, and/or, that the operating temperature of the process of the present invention is at least 10° C. above the dew point temperature, of both (i) the feedstock to the process; and (ii) of the product composition of the process. The product composition of the process of the present invention (i.e. (ii)) is dependent on factors such as the initial feed composition and the degree of conversion within the reactor.

For the purposes of the present invention, the term “dew point temperature” is defined as being the threshold temperature at which a dry gas exists for a given pressure; for example, for a given mixture at a given pressure, if the system temperature is raised to above the dew point temperature, the mixture will exist as a dry gas; likewise below the dew point temperature, the mixture will exist as a vapour containing some liquid. Similarly, the term “dew point pressure” is defined as being a threshold pressure at which a dry gas exists for a given temperature; for example, for a given mixture at a given temperature, if the system pressure is below the dew point pressure, the mixture will exist as a dry gas; above the dew point pressure, the mixture will exist as a vapour containing liquid.

The temperature at which the process of the present invention is operated is at least 210° C., preferably at least 220° C., more preferably at least 230° C. and most preferably at least 240° C.; and is at most 270° C., preferably at most 265° C., more preferably at most 260° C., even more preferably at most 255° C., and most preferably at most 250° C.

The process of the present invention is operated at a pressure in the range of from 1.5 MPa to 2.5 MPa; preferably at a pressure in the range of from 1.6 MPa to 2.4 MPa.

The preferred reaction conditions used in the process of the present invention are such that the dehydration process is run at moderate conversion of the feedstock comprising ethanol to olefins. For the purposes of the present invention, moderate conversion of the feedstock comprising ethanol to olefins is defined as being the conversion of alcohols (for example the ethanol and optionally propanol) and/or their corresponding derived ethers (for example diethyl ether) into the corresponding olefins (e.g. ethylene and optionally propylene), and means that from 10 to 80%, more preferably from 20 to 60%, of the alcohols and/or ethers are converted per pass.

In a preferred embodiment of the present invention, any unconverted alcohols and/or ethers (which may be present in the feedstock or produced in the process of the present invention) present in the product stream resulting from the process of the present invention are recycled back to the inlet of the reactor. Therefore, in a preferred embodiment of the present invention, the feedstock comprising ethanol additionally contains a recycle stream comprising alcohols and ethers. Said recycle stream typically contains unconverted alcohols, ethers (either unconverted ethers that may be present in the feedstock or ethers produced during the dehydration process) and water. Any suitable means of recycling the unconverted alcohols and/or ethers present in the product stream resulting from the process of the present invention may be used.

The feedstock used in the process of the present invention is one that comprises ethanol; optionally the feedstock may also comprise water and other components.

The feedstock used in the process of the present invention preferably contains less than 10 wt %, more preferably less than 2 wt %, of propanol. Preferably, the feedstock used in the process of the present invention has an iso-propanol content of less than 5 wt %, more preferably less than 1 wt %, even more preferably less than 0.1 wt %, and most preferably contains no iso-propanol.

The feedstock used in the process of the present invention may also additionally comprise homo- and/or mixed-ethers of ethanol, propanol and iso-propanol; for example: diethyl ether, di-n-propyl ether, ethyl n-propyl ether, ethyl isopropyl ether, n-propyl isopropyl ether, di-iso-propyl ether and mixtures thereof. In one embodiment of the process of the present invention, the ethers that may be present in the feedstock comprising ethanol may be present in a recycle stream that is contained within the feedstock; alternatively, the ethers that may present in the feedstock may derive from sources other than a recycle stream.

Thus, in a preferred embodiment of the present invention, the feedstock contains up to 80 wt % of homo- and/or mixed-ethers of ethanol, propanol and iso-propanol; more preferably the feedstock contains up to 50 wt % of homo- and/or mixed-ethers of ethanol, propanol and iso-propanol. In one embodiment of the present invention, the feedstock contains at least 5 wt % of homo- and/or mixed-ethers of ethanol, propanol and iso-propanol, preferably at least 10 wt % of homo- and/or mixed-ethers of ethanol, propanol and iso-propanol.

In a particularly preferred embodiment of the present invention, the feedstock used in the process of the present invention contains up to 80 wt % of diethyl ether, more preferably up to 50 wt % of diethyl ether. In this embodiment of the present invention, the feedstock used in the process of the present invention preferably contains at least 5 wt % of diethyl ether, more preferably at least 10 wt % of diethyl ether.

The presence of alcohols containing four or more carbon atoms in a feedstock comprising ethanol which is to be dehydrated using a heteropolyacid can lead to an increase in the amount of C4 compounds produced. Therefore, in a preferred embodiment of the present invention, the feedstock comprising ethanol has a total content of alcohols containing four or more carbon atoms of less than 5 wt %, more preferably less than 1 wt %, even more preferably less than 0.1 wt %, and most preferably the feedstock comprising ethanol contains no alcohols containing four or more carbon atoms.

The presence of methanol in a feedstock comprising ethanol which is to be dehydrated using a heteropolyacid could lead to various undesirable side reactions, such as MTO (methanol to olefins) reactions, formation of methyl ethers, and the alkylation of olefins. Therefore, it is preferred that the feedstock comprising ethanol has a methanol content of less than 5 wt %, more preferably less than 2 wt %, even more preferably less than 0.5 wt %, and most preferably there is no methanol.

Typically, the feedstock comprising ethanol used in the process of the present invention will contain at least 5 wt % ethanol, preferably at least 10 wt % ethanol, more preferably at least 15 wt % ethanol, and most preferably at least 20 wt % ethanol.

The feedstock comprising ethanol used in the process of the present invention may contain substantial amounts of water; for examples the feedstock used in the process of the present invention may contain up to 50 wt % water. Preferably, the feedstock used in the process of the present invention contains at most 25 wt % water, more preferably at most 20 wt % water. However, due to the heat of vaporization and heat capacity of water, it may be desirable to operate the process of the present invention using a feedstock which contains lower levels of water. Thus, in a particularly preferred embodiment, the feedstock comprising ethanol used in the process of the present invention contains at most 10 wt % water, more preferably at most 5 wt % water.

Since the presence of water in the feedstock is believed to have a beneficial effect on the stability and/or performance of heteropolyacid catalysts in the dehydration of alcohols, according to a particularly preferred embodiment of the present invention, the feedstock to the process of the present invention contains at least 0.1 wt % water, more preferably at least 0.5 wt % water, most preferably at least 1 wt % water.

The source of the feedstock comprising ethanol is not critical to the present invention, for example the feedstock comprising ethanol may be produced by the fermentation of, for example, sugars, starches and/or cellulosic materials, or alternatively may be produced from synthesis gas.

If the feedstock comprising ethanol is produced from synthesis gas, the process of the present invention may be used in a process to produce ethylene from hydrocarbons.

For example, at least part of the feedstock comprising ethanol may be a composition comprising ethanol prepared from a feed stream comprising hydrocarbons by a process comprising the following steps:
(a) preparing a mixture of carbon oxide(s) and hydrogen from the feed stream comprising hydrocarbons in a synthesis gas reactor, and
(b) converting said mixture of carbon oxide(s) and hydrogen from step (a) in the presence of a suitable particulate catalyst in a reactor at a temperature in the range of from 200 to 400° C. and at a pressure in the range of from 5 to 20 MPa, into a composition comprising ethanol.

Thus, the present invention can also provide a process for the conversion of hydrocarbons to ethylene comprising the steps of:
(a) preparing a mixture of carbon oxide(s) and hydrogen from a feed stream comprising hydrocarbons in a synthesis gas reactor;
(b) converting said mixture of carbon oxide(s) and hydrogen from step (a) in the presence of a suitable particulate catalyst in a reactor at a temperature in the range of from 200 to 400° C. and at a pressure in the range of from 5 to 20 MPa, into a composition comprising ethanol; and
(c) using at least part of said composition comprising ethanol as at least part of a feedstock comprising ethanol to produce ethylene, in the presence of a phosphotungstic acid catalyst, by a process as described herein.

For the purpose of the above embodiment, any hydrocarbon containing feed stream that can be converted into a composition comprising carbon monoxide and hydrogen (e.g. a synthesis gas (or “syngas”) composition) may be used.

The hydrocarbon used in the preparation of the mixture of carbon oxide(s) and hydrogen in step (a) of the embodiments described above is preferably a carbonaceous material, for example biomass, plastic, naphtha, refinery bottoms, smelter off gas, municipal waste, coal, coke and/or natural gas; with coal and natural gas being preferred, and natural gas being most preferred.

The mixture of carbon oxide(s) and hydrogen (e.g. synthesis gas), may undergo purification prior to being fed to any reaction zones in step (b) of the embodiments described above. The purification of the mixture of carbon oxide(s) and hydrogen (e.g. synthesis gas purification) may be carried out by processes known in the art. See, for example, Weissermel, K. and Arpe H.-J., Industrial Organic Chemistry, Second, Revised and Extended Edition, 1993, pp. 19-21.

The present invention also provides the use of a phosphotungstic acid in a process for the preparation of ethylene from a feedstock comprising ethanol, for providing a reduced selectivity toward C4 hydrocarbon compounds compared to when silicotungstic acid based catalysts are used under the same process conditions.

The process of the present invention is illustrated in the following examples.

### EXAMPLES

### Support Material

The support materials used in the examples were CariAct® Q15 silica pellets (ex. Fuji Silysia) and Davicat® Grade 57 silica granules (ex. Grace Davison).

The surface area, pore volume and mean pore size diameter (PSD) of the support materials were analysed using nitrogen porosimetry and are recorded in Table 1 below.

TABLE 1

| | Surface Area | Pore Volume | Mean PSD |
|---|---|---|---|
| Support | (m²/g) | (cm³/g) | (Å) |
| CariAct Q15 | 208 | 1.02 | 196 |
| Davicat Grade 57 | 284 | 1.11 | 156 |

### Heteropolyacids

The heteropolyacids used in the preparation of the catalysts employed in the following examples were silicotungstic acid (H₄[SiW₁₂O₄₀].24H₂O; Mw 3310.6) and phosphotungstic acid (H₃[PW₁₂O₄₀].24H₂O; Mw 3312.4). The silicotungstic acid and phosphotungstic acid used in the preparation of catalysts A and B were obtained from Aldrich, and the silicotungstic acid and phosphotungstic acid used in the preparation of catalysts C to G were obtained from Nippon Inorganic Chemicals.

### Catalyst Preparations

The catalysts used in the following examples were prepared by impregnating the support material using an aqueous heteropolyacid solution. The aqueous heteropolyacid acid solution was prepared by dissolving a weighed amount of the heteropolyacid in distilled water. To this acid solution was added a weighed amount of the support material. The support material was left to soak in the acid for approximately 1 hr with occasional agitation to dislodge any air bubbles that may have been trapped. After soaking, the catalyst (i.e. the impregnated support material) was removed from the solution by filtration and allowed to drain until no more liquid was being removed from the catalyst. After draining was complete, the catalyst was transferred to a ceramic tray and dried in a muffle furnace at 130° C. under nitrogen.

The dried catalyst was weighed and the amount of heteropolyacid adsorbed on the catalyst was calculated from the difference in weight of the catalyst versus the weight of the support material.

The details of the support material and heteropolyacids used to prepare the catalysts used in the following examples, and the calculated heteropolyacid loading of the catalysts, is provided in Table 2 below.

TABLE 2

| | | Weight of | | Weight of HPA | Weight of water for | Weight | HPA | HPA Loading |
|---|---|---|---|---|---|---|---|---|
| Catalyst | Support | Support (g) | HPA Type | in solution (g) | HPA solution (g) | catalyst (g) | absorbed (g) | (g/kg catalyst) |
| A | G57 | 10.1 | SiW | 10.1 | 22.1 | 14.1 | 4.0 | 286 |
| B | G57 | 10.1 | PW | 10.0 | 22.9 | 13.8 | 3.7 | 271 |
| C | Q15 | 200.0 | SiW | 200.1 | 453.8 | 270.8 | 70.8 | 261 |
| D | G57 | 30.0 | SiW | 7.1 | 75.6 | 32.5 | 2.5 | 76 |
| E | G57 | 30.2 | SiW | 2.0 | 75.5 | 30.7 | 0.5 | 16 |
| F | G57 | 10.0 | SiW | 10.2 | 22.1 | 13.9 | 3.9 | 279 |
| G | G57 | 10.1 | PW | 10.3 | 22.0 | 14.3 | 4.2 | 292 |

HPA = Heteropolyacid
SiW = Silicontungstic acid (H₄[SiW₁₂O₄₀]•24H₂O)
PW = phosphotungstic acid (H₃[PW₁₂O₄₀]•24H₂O)
Note.
When calculating the quantity of heteropolyacid (HPA) used for the catalyst preparation and when calculating the amount of HPA adsorbed on the catalyst it was assumed that the heteropolyacid was fully hydrated and was present as the 24 hydrate compound.

### Catalyst Testing

Catalysts A to G detailed in Table 2 above, were each, independently, crushed using a mortar and pestle, with the particles having a particle size of 125 to 180 μm being separated from the resulting broken catalyst using a series of stacked sieves consisting of a base, a 125 μm mesh sieve and a 180 μm mesh sieve.

Approximately 1 ml of the 125 to 180 μm catalyst particles were, independently, loaded into separate reactor tubes (internal diameter: 4.2 mm) of a parallel flow reactor (catalyst volumes in each reactor varied from 0.776 to 1.164 ml).

The reactors were pressure tested and then heated to 220° C. under a nitrogen flow.

A liquid feed of ethanol, diethyl ether and water was vapourised and mixed with nitrogen, and said feed was introduced to the reactors once the temperature of the reactor had reached 220° C. Methane, a compound not produced or consumed in the process, was also introduced into the reactor and was used as an internal standard to enable accurate measurement of product rates exiting the reactors.

The feed introduced into the reactors consisted of ethanol (28% v/v), diethyl ether (34.5% v/v), water (3.3% v/v), nitrogen (32.7% v/v) and methane (1.5% v/v); and was introduced into the reactor at a pressure of 20 barg. The rate at which each of the components were delivered to the reactor was: N₂—1.001 l/hr; ethanol—1.724 g/hr; diethyl ether—3.417 g/hr; methane—0.032 g/hr; water—0.080 g/hr.

The catalysts were then sequentially tested under the following sequence of temperatures: (a) 220° C. for 24 hrs to achieve steady state performance; (b) 210° C. for 24 hrs; (c) 230° C. for 24 hrs; (d) 240° C. for 24 hrs; and finally (e) 220° C. for 24 hrs.

The composition of the product streams from each of the reactors was analysed by gas chromatography, and the data from the latter four test periods, (b) to (e), is recorded in ascending temperature order in Table 3 below.

TABLE 3

| | | | | C4 | Average C4 |
|---|---|---|---|---|---|
| | | Reactor | Ethylene | Selectivity | selectivity |
| | | Temper- | Productivity | relative to | relative to |
| Exam- | Cata- | ature | (g ethylene/l | ethylene | ethylene |
| ple | lyst | (° C.) | catalyst/hr) | (ppm wt) | productivity |
| A* | A | 210 | 475 | 373 | 0.7357 |
| B* | A | 220 | 869 | 615 | |
| C* | A | 230 | 1336 | 926 | |
| D* | A | 240 | 1925 | 1461 | |
| 1 | B | 210 | 315 | 221 | 0.6257 |
| 2 | B | 220 | 630 | 437 | |
| 3 | B | 230 | 1004 | 624 | |
| 4 | B | 240 | 1486 | 910 | |
| E* | C | 210 | 380 | 324 | 0.7081 |
| F* | C | 220 | 734 | 523 | |
| G* | C | 230 | 1182 | 778 | |
| H* | C | 240 | 1699 | 1231 | |
| I* | D | 210 | 100 | 166 | 1.2215 |
| J* | D | 220 | 263 | 361 | |
| K* | D | 230 | 538 | 688 | |
| L* | D | 240 | 978 | 1163 | |
| M* | E | 210 | 12 | C4s too low | 2.571 |
| N* | E | 220 | 30 | for | |
| O* | E | 230 | 58 | quantification | |
| P* | E | 240 | 113 | 290 | |
| Q* | F | 210 | 457 | 339 | 0.67 |
| R* | F | 220 | 864 | 580 | |
| S* | F | 230 | 1362 | 845 | |
| T* | F | 240 | 1949 | 1345 | |
| 5 | G | 210 | 348 | 205 | 0.4999 |
| 6 | G | 220 | 712 | 415 | |
| 7 | G | 230 | 1178 | 589 | |
| 8 | G | 240 | 1740 | 839 | |

*Comparative

The C4 hydrocarbons detected were i-butane, 1-butene, trans-2-butene, cis-2-butene.

The C4 selectivity is the total weight of C4 compounds in the product composition relative to the total weight of ethylene in the product composition.

The average C4 selectivity relative to ethylene productivity is the gradient of the line of best fit when the C4 selectivity is plotted against the ethylene productivity (see FIGS. 1 and 2).

FIGS. 1 and 2 plot the C4 selectivity (ppmw) against the ethylene productivity (ethylene (g)/catalyst(1)/hr) for the phosphotungstic acid catalysts (B and G) and the silicotungstic acid catalysts (A, C, D, E and F).

1-11. (canceled)

12. Process for the preparation of ethylene comprising contacting a feedstock comprising ethanol with a phosphotungstic acid catalyst at a temperature in the range of from 210° C. to 270° C. and a pressure in the range of from 1.5 MPa to 2.5 MPa, wherein the phosphotungstic acid catalyst is a supported phosphotungstic acid catalyst and wherein the performance of the phosphotungstic acid catalyst under test Conditions A satisfies the following inequality:
$$\frac{\begin{matrix}Selectivity towards C 4 \\ compounds in ethylene ( ppmw )\end{matrix}}{ppmw} \leq \frac{0.6257 \times Ethylene productivity ( g / l / hr )}{g / l / hr}$$
where test Conditions A are:
a tubular plug flow reactor having an internal reactor diameter of 4.2 mm containing a catalyst volume of about 1 cm³;
a catalyst particle size of from 125 to 180 μm;
a phosphotungstic acid loading in the range of from 270 to 295 g of phosphotungstic acid per kg catalyst;
a temperature of 240° C. and a pressure of 2 MPa; and
feed flow rates as follows: ethanol (1.724 g/hr), diethylether (3.417 g/hr), water (0.080 g/hr), nitrogen (1.001 g/hr) and methane (0.032 g/hr).

13. Process according to claim 12, wherein the phosphotungstic acid has a molecular weight in the range of from 700 to 8500.

14. Process according to claim 12, wherein the phosphotungstic acid catalyst is selected from:
18-tungstophosphoric acid—H₆[P₂W₁₈O₆₂].xH₂O
12-tungstophosphoric acid—H₃[PW₁₂O₄₀].xH₂O
and the partial salts, or mixtures thereof.

15. Process according to claim 14, wherein the phosphotungstic acid is selected from:
18-tungstophosphoric acid—H₆[P₂W₁₈O₆₂].xH₂O
12-tungstophosphoric acid—H₃[PW₁₂O₄₀].xH₂O
and the partial salts:
Monopotassium tungstophosphate—KH₂[PW₁₂O₄₀].xH₂O
Monoammonium tungstophosphate—[NH₄]H₂[PW₁₂O₄₀].xH₂O
Monosodium tungstophosphate—NaH₂[PW₁₂O₄₀].xH₂O
Monocesium tungstophosphate—CsH₂[PW₁₂O₄₀].xH₂O
Monopotassium salt of 18-tungstophosphoric acid—KH₅[P₂W₁₈O₆₂].xH₂O
Monoammonium salt of 18-tungstophosphoric acid—[NH₄]H₅[P₂W₁₈O₆₂].xH₂O
Monosodium salt of 18-tungstophosphoric acid—NaH₅[P₂W₁₈O₆₂].xH₂O
Monoscesium salt of 18-tungstophosphoric acid—CsH₅[P₂W₁₈O₆₂].xH₂O
on mixtures thereof.

16. Process according to claim 12, wherein the phosphotungstic acid is
12-tungstophosphoric acid—H₃[PW₁₂O₄₀].xH₂O

17. Process according to claim 12, wherein the temperature and pressure at which the feedstock comprising ethanol is contacted with the phosphotungstic acid catalyst are selected such that the process is operated in a vapour phase state.

18. Process according to claim 17, wherein the pressure is at least 0.1 MPa below the dew point pressure, and/or, the temperature is at least 10° C. above the dew point temperature, of both the feedstock comprising ethanol and the product composition of the process.

19. Process according to claim 12, wherein the temperature at which the feedstock comprising ethanol is contacted with the phosphotungstic acid catalyst is in the range of from 220° C. to 260° C.

20. Process according to claim 12, wherein the pressure at which the feedstock comprising ethanol is contacted with the phosphotungstic acid catalyst is in the range of from 1.6 MPa to 2.4 MPa.

21. Process according to claim 12, wherein at least part of the feedstock comprising ethanol may be a composition comprising ethanol prepared from a feed stream comprising hydrocarbons by a process comprising the following steps:
(a) preparing a mixture of carbon oxide(s) and hydrogen from the feed stream comprising hydrocarbons in a synthesis gas reactor, and
(b) converting said mixture of carbon oxide(s) and hydrogen from step (a) in the presence of a suitable particulate catalyst in a reactor at a temperature in the range of from 200 to 400° C. and at a pressure in the range of from 5 to 20 MPa, into a composition comprising ethanol.
